(19) United States
(12) Patent Application Publication
(10) Pub. No.: US 2005/0032810 A1
(43) Pub. Date: Feb. 10, 2005

(54) Novel anthelmintic and insecticidal compositions

(76) Inventors: Byung Hyun Lee, Chelsea, MI (US); Martha Jane Larsen, South Lyon, MI (US); Teresa Maria Kubiak, Richland, MI (US)

Correspondence Address: PHARMACIA & UPJOHN, 301 HENRIETTA ST, 0228-32-LAW, KALAMAZOO, MI, 49007, US

(21) Appl. No.: 10/912,513

(22) Filed: Aug. 5, 2004

Related U.S. Application Data
(60) Provisional application No. 60/493,296, filed on Aug. 7, 2003.

(51) Int. Cl. A61K 31/506; C07D 43/04

(52) U.S. Cl. 514/252.14; 514/275; 544/295; 544/331

(57) ABSTRACT

Novel anthelmintic and insecticidal compositions containing pyrimidine derivatives as active ingredients are disclosed.

### CROSS-REFERENCE TO RELATED APPLICATION

This application claims the benefit of U.S. provisional application Ser. No. 60/493,296, filed Aug. 7, 2003, under 35 USC 119(e)(1), which is incorporated herein by reference.

### FIELD OF THE INVENTION

The present invention relates to novel anthelmintic and insecticidal compositions in general, and, more specifically, compositions containing pyrimidine derivatives as active ingredients.

### TECHNOLOGY DESCRIPTION

Control of parasitic infections in human and animal populations remains an important global endeavor. The causative organisms may be categorized as endoparasitic members of the classes Nematoda, Cestoidea and Trematoda or phylum Protozoa, or as ectoparasitic members of the phylum Arthropoda. These organisms cause infections of the stomach, intestinal tracts, lymphatic system, tissues, liver, lungs, heart and brain. Examples include trichinosis, lymphatic filariasis, onchocerciasis, schistosomiasis, leishmaniasis, trypanosomiasis, giardiasis, coccidiosis and malaria. The ectoparasites of the phylum arthropoda include lice, ticks, mites, biting flies, fleas and mosquitoes. These often serve as vectors and intermediate hosts to endoparasites for transmission to human or animal hosts. While certain helminthiases can be treated with known drugs, evolutionary development of resistance necessitates a further search for improved efficacy in next generation anthelmintic agents.

The control of ectoparasites, such as fleas, ticks, biting flies and the like, has long been recognized as an important aspect of human and animal health regimens. Traditional treatments were topically applied, such as the famous dips for cattle, and indeed such treatments are still in wide use. The more modern thrust of research, however, has been towards compounds which can be administered orally or parenterally to the animals and which will control ectoparasitic populations by poisoning individual parasites when they ingest the blood of a treated animal.

The control of endoparasites, especially intestinal parasites, has also been an important aspect of human and animal health regimens. Although a number of ectoparasiticides and endoparasiticides are in use, these suffer from a variety of problems, including a limited spectrum of activity, the need for repeated treatment and, in many instances, resistance by parasites. The development of novel endo- and ectoparasiticides is therefore essential to ensure safe and effective treatment of a wide range of parasites over a long period of time.

Despite the above teachings, there still exists a need in the art for treatment of pests.

### BRIEF SUMMARY OF THE INVENTION

In accordance with the present invention, a novel composition of matter that is capable of treatment of pests is provided. The composition contains pyrimidine derivatives of Formula I: 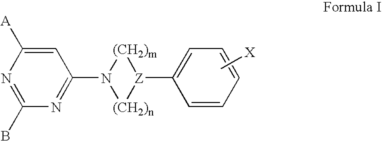
wherein A and B are independently selected from the group comprising H, C₁₋₆ alkyl, NRR₁, and C₁₋₆ alkoxy;
X is one to three substituents selected from the group comprising halogen, C₁₋₆ alkyl, —OH, C₁₋₆ alkoxy, C₁₋₆ alkylthio, —NRR₁, —CO₂R, —C(O)NRR₁, —CN, —NO₂ and —SO₂NRR₁;
R and R₁ are independently H, C₁₋₆ alkyl, or taken together with the N to which they are attached form a 5-7 membered ring optionally containing an additional heteroatom of NR, O or S;
m is 1 or 2;
n is 1, 2or 3;
when m and n are both 2, Z is N, CH or C—O—R;
when m and n are not both 2, Z is CH or C—O—R; as active ingredients.

### DETAILED DESCRIPTION OF THE EMBODIMENTS

Definitions

In describing the embodiments, certain terminology will be utilized for the sake of clarity. Such terminology is intended to encompass the recited embodiments, as well as all technical equivalents that operate in a similar manner for a similar purpose to achieve a similar result.

The term “alkyl”, by itself or as part of another substituent, means, unless otherwise stated, a straight or branched chain, or cyclic hydrocarbon radical, or combination thereof, which may be fully saturated, mono- or polyunsaturated and can include di- and multivalent radicals, having 1 to 6 carbon atoms. Examples of saturated hydrocarbon radicals include groups such as methyl, ethyl, n-propyl, isopropyl, n-butyl, t-butyl, isobutyl, sec-butyl, cyclohexyl, (cyclohexyl)ethyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, I- and 3-propynyl, 3-butynyl, and the higher homologs and isomers. A “lower alkyl” is a shorter chain alkyl or group, having eight or fewer carbon atoms.

The terms “alkoxy, alkylamino” and “alkylthio” refer to those groups having an alkyl group attached to the remainder of the molecule through an oxygen, nitrogen or sulfur atom, respectively. Similarly, the term “dialkylamino” is used in a conventional sense to refer to —NR₁R₂ wherein the R₁ and R₂ groups can be the same or different alkyl groups.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl. include cyclopentyl, cyclohexyl, 1-cyclohexenyl, 3-cyclohexenyl, cycloheptyl, and the like. Examples of heterocycloalkyl include 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The terms “halo” or “halogen,” by themselves or as part of another substituent, mean, unless otherwise stated, a fluorine, chlorine, bromine, or iodine atom. Additionally, terms such as “fluoroalkyl,” are meant to include monofluoroalkyl and polyfluoroalkyl.

As used herein, the term “heteroatom” is meant to include oxygen (O), nitrogen (N) and sulfur (S).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt.

When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactouronic acids and the like (see, for example, Berge et al. (1977) J. Miami. Sci., 66:1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds that are in a prodrug form. The expression “prodrug” denotes a derivative of a known direct acting drug, which derivative has enhanced delivery characteristics and therapeutic value as compared to the drug, and is transformed into the active drug by an enzymatic, for example by hydrolysis in blood, or chemical process [see T. Higuchi and V. Stella, “Pro-drugs as Novel Delivery Systems,” Vol. 14 of the A.C.S. Symposium Series; Bioreversible Carriers in Drug Design, ed. Edward B. Roche, American Pharmaceutical Association and Pergamon Press, (1987); Notari, R. E., “Theory and Practice of Prodrug Kinetics,” Methods in Enzymology, 112:309-323 (1985); Bodor, N., “Novel Approaches in Prodrug Design,” Drugs of the Future, 6(3):165-182 (1981); and Bundgaard, H., “Design of Prodrugs: Bioreversible-Derivatives for Various Functional Groups and Chemical Entities,” in Design of Prodrugs (H. Bundgaard, ed.), Elsevier, N.Y. (1985)]. The prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). As used herein, a “prodrug” is any covalently bonded carrier that releases in vivo the active parent drug according to the Formula I when such prodrug is administered to the subject. Prodrugs of the compounds of Formula I are prepared by modifying functional groups present on the compound in such a way that the modifications are cleaved, either in routine manipulation or in vivo, to the parent compound. Prodrugs include, but are not limited to, compounds derived from compounds of Formula I wherein hydroxy, amine or sulfhydryl groups are bonded to any group that, when administered to the subject, cleaves to form the free hydroxyl, amino or sulfhydryl group, respectively. Selected examples include, but are not limited to, biohydrolyzable amides and biohydrolyzable esters and biohydrolyzable carbamates, carbonates, acetate, formate and benzoate derivatives of alcohol and amine functional groups. Furthermore, prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of Formula I and Formula II. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

### DETAILED DESCRIPTION OF THE INVENTION

One embodiment of present invention provides a compound of formula I: 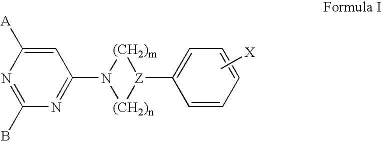
wherein A and B are independently selected from the group comprising H, C₁₋₆ alkyl, NRR₁, and C₁₋₆ alkoxy;
X is one to three substituents selected from the group comprising halogen, C₁₋₆ alkyl, —OH, C₁₋₆ alkoxy, C₁₋₆ thioalkoxy, —NRR₁, —CO₂R, —CONRR₁, —CN, —NO₂ and —SO₂NRR₁;
R and R₁ are independently H, C₁₋₆ alkyl, or taken together with the N to which they are attached form a 5-7 membered ring optionally containing an additional heteroatom of NR, O or S;
m is 1 or 2;
n is 1, 2 or 3;
when m and n are both 2, Z is N, CH or C—O—R;
when m and n are not both 2, Z is CH or C—O—R;
or pharmaceutically acceptable salts thereof.

A second embodiment of the present invention provides a composition comprising a compound of Formula I. Another embodiment of the present invention comprises a compound of Formula I and a carrier.

Another embodiment of the present invention comprises a process for the treatment or prevention of parasitic diseases in mammals, including humans, plants or agricultural crops comprising the step of administering to the mammal, plant or crop an effective amount of the above composition.

A further embodiment of the present invention comprises the use of the above-described composition to prepare a medicament for the treatment or prevention of parasitic diseases in mammals.

Yet another embodiment of the present invention comprises the above-described composition for use as a medicament.

An object of the present invention is to provide novel compositions that can be broadly used against parasites.

Still another object of the present invention is to provide a method for preventing or treating parasitic diseases in mammals by using a novel composition.

A further object of the present invention is to provide a method for producing a medicament using a novel composition.

These, and other objects, will readily be apparent to those skilled in the art.

In practice, the amount of the compound to be administered ranges from about 0.001 to 100 mg. per kg. of animal body weight, such total dose being given at one time or in divided doses over a relatively short period of time such as 1-5 days. Control of such parasites is obtained in animals by administering from 0.02 to 30 mg. per kg. of body weight in a single dose. Repeat treatments are given as required to combat re-infections and are dependent upon the species of parasite and the husbandry techniques being employed. The techniques for administering these materials to animals are known to those skilled in the veterinary field.

For use as an antiparasitic agent in animals the inventive composition may be administered internally either orally or by injection, or topically as a liquid drench or as a shampoo. These compositions may be administered orally in a unit dosage form such as a capsule, bolus or tablet. The capsules and boluses comprise the active ingredients admixed with a carrier vehicle such as starch, talc, magnesium stearate, or di-calcium phosphate. The drench is normally a solution, suspension or dispersion of the active ingredients usually in water together with a suspending agent such as bentonite and a wetting agent or like excipient. Generally, the drenches also contain an antifoaming agent. Drench formulations generally contain from about 0.01 to 10% by weight of each active compound. Preferred drench formulations may contain from 0.05 to 5.0% of each active by weight.

Where it is desired to administer the inventive composition in a dry, solid unit dosage form, capsules, boluses or tablets containing the desired amount of active compounds usually are employed. These dosage forms are prepared by intimately and uniformly mixing the active ingredient with suitable finely divided diluents, fillers, disintegrating agents and/or binders such as starch, lactose, talc, magnesium stearate, vegetable gums and the like. Such unit dosage formulations may be varied widely with respect to their total weight and content of the antiparasitic agent depending upon factors such as the type of host animal to be treated, the severity and type of infection and the weight of the host.

When the active composition is to be administered via an animal feedstuff, it is intimately dispersed in the feed or used as a top dressing or in the form of pellets which may then be added to the finished feed or optionally fed separately. Alternatively, the antiparasitic compositions of the present invention may be administered to animals parenterally, for example, by intraruminal, intramuscular, intratracheal, or subcutaneous injection in which event the active ingredients are dissolved or dispersed in a liquid carrier vehicle. For parenteral administration, the active materials are suitably admixed with an acceptable vehicle, preferably of the vegetable oil variety such as peanut oil, cottonseed oil and the like. Other parenteral vehicles such as organic preparation using solketal, propylene glycol, glycerol formal, and aqueous parenteral formulations are also used, often in combination in various proportions. Still another carrier which can be selected is either N-methylpyrrolidone or 2-pyrrolidone and mixtures of the two. This formulation is described in greater detail in U.S. Pat. No. 5,773,442. To the extent necessary for completion, this patent is expressly incorporated by reference. The active compound or compounds are dissolved or suspended in the parenteral formulation for administration; such formulations generally contain from 0.005 to 5% by weight of each active compound.

In an example of the embodiment, the carrier contains propylene glycol (1-99 percent by weight of the carrier) and glycerol formal (99-1 percent by weight of the carrier), with the relative amounts being 60% propylene glycol and 40% glycerol formal.

The present compositions may also be useful in yet another method in which the same active agents as above defined are employed as a “feed through larvicide.” In this method, the compound is administered to a vertebrate animal, especially a warm-blooded animal, in order to inhibit parasitic organisms that live in the feces of the animal. Such organisms are typically insect species in the egg or larval stage.

The inventive compositions are primarily useful as antiparasitic agents for the treatment and/or prevention of helminthiasis in all mammals, which includes, but is not limited to, humans, cattle, sheep, deer, horses, dogs, cats, goats, swine, and birds. They are also useful in the prevention and treatment of parasitic infections of these mammals by ectoparasites such as ticks, mites, lice, fleas and the like. In treating such infections the inventive compositions may be used individually or in combination with each other or with other unrelated antiparasitic agents.

The exact dosage and frequency of administration of the inventive compositions depend on many factors, including (but not limited to) the severity of the particular condition being treated, the age, weight, and general physical condition of the particular patient (human or animal), and other medication the patient may be taking. These factors are well known to those skilled in the art, and the exact dosage and frequency of administration can be more accurately determined by measuring the concentration of the inventive composition in the patient's blood and/or the patient's response to the particular condition being treated.

The inventive compositions may also be used to combat agricultural pests that attack crops either in the field or in storage. The inventive compositions are applied for such uses as sprays, dusts, emulsions and the like either to the growing plants or the harvested crops. The techniques for applying the inventive compositions in this manner are known to those skilled in the agricultural arts.

Accordingly, it can be seen that the present methods can be utilized for protection against a wide range of parasitic organisms. Further, it should be noted that protection is achieved in animals with existing parasitic infections by eliminating the existing parasites, and/or in animals susceptible to attack by parasitic organisms by preventing parasitic attack. Thus, protection includes both treatment to eliminate existing infections and prevention against future infestations.

Representative parasitic organisms include the following:
Platyhelminthes:
   Trematoda such as
      Clonorchis
      Echinostoma
      Fasciola hepatica (liver fluke)
      Fasciola gigantica
      Fascioloides magna
      Fasciolopsis
      Metagonimus
      Paragonimus
      Schistosoma spp.
   Nemathelminthes:
      Ancylostoma
      Angiostrongylus
      Anisakis
      Ascaris
      Brugia
      Bunostomum
      Cooperia
      Cyathostomum
      Cylicocyclus
      Dictyocaulus (lungworm)
      Dipetalonema
      Dirofilaria (heartworm)
      Dracunculus
      Elaeophora
      Gaigeria
      Globocephalus urosubulatus
      Haemonchus
      Metastrongylus (lungworm)
      Muellerius (lungworm)
      Necator americanus
      Nematodirus
      Oesophagostomum
      Onchocerca
      Ostertagia
      Parascaris
      Protostrongylus (lungworm)
      Setaria
      Stephanofilaria
      Syngamus
      Teladorsagia
      Toxascaris
      Toxocara
      Trichinella
      Trichostrongylus
      Uncinaria stenocephala
      Wuchereria bancrofti
Arthropoda:
   Crustacea:
      Argulus
      Caligus
   Arachnida:
      Amblyomma americanum (Lone-star tick)
      Amblyomma maculatum (Gulf Coast tick)
      Argas persicus (fowl tick)
      Boophilus microplus (cattle tick)
      Demodex bovis (cattle follicle mite)
      Demodex canis (dog follicle mite)
      Dermacentor andersoni (Rocky Mountain spotted fever tick)
      Dermacentor variabilis (American dog tick)
      Dermanyssus gallinae (chicken mite)
      Ixodes ricinus (common sheep tick)
      Knemidokoptes gallinae (deplumming mite)
      Knemidokoptes mutans (scaly-leg mite)
      Otobius megnini (ear tick)
      Psoroptes equi (scab mite)
      Psoroptes ovis (scab mite)
      Rhipicephalus sanguineus (brown dog tick)
      Sarcoptes scabiei (mange mite)
   Insecta:
      Aedes (mosquito)
      Anopheles (mosquito)
      Culex (mosquito)
      Culiseta (mosquito)
      Bovicola bovis (cattle biting louse)
      Callitroga hominivorax (blowfly)
      Chrysops spp. (deer fly)
      Cimex lectularius (bed bug)
      Ctenocephalis canis (dog flea)
      Ctenocephalis fells (cat flea)
      Culicoides spp. (midges, sandflies, punkies, or no-see-ums)
      Damalinia ovis (sheep biting louse)
      Dermaobia spp. (warble fly)
      Dermatophilus spp. (fleas)
      Gasterophilus haemorrhoidalis (nose bot fly)
      Gasterophilus intestinalis (common horse bot fly)
      Gasterophilus nasalis (chin fly)
      Glossina spp. (tsetse fly)
      Haematobia irritans (horn fly, buffalo fly)
      Haematopinus asini (horse sucking louse)
      Haematopinus eurysternus (short nosed cattle louse)
      Haematopinus ovilius (body louse)
      Haematopinus suis (hog louse)
      Hydrotaea irritans (head fly)
      Hypoderma bovis (bomb fly)
      Hypoderma lineatum (heel fly)
      Linognathus ovillus (body louse)
      Linognathus pedalis (foot louse)
      Linognathus vituli (long nosed cattle louse)
      Lucilia spp. (maggot fly)
      Melophagus ovinus (sheep ked)
      Oestrus ovis (nose hot fly)
      Phormia regina (blowfly)
      Psorophora
      Reduviid bugs (assassin bug)
      Simulium spp. (black fly)
      Solenopotes capillatus (little blue cattle louse)
      Stomoxys calcitrans (stable fly)
      Tabanus spp. (horse fly)
         Parasitic organisms that live in feces are typically the egg and larval stages of insects such as:

Musca domestica (housefly)

Musca autumnalis (face fly)

Haematobia spp. (horn fly, buffalo fly and others).

Non-limiting examples of the invention include

a) 6-(4-phenylpiperidin-1-yl)pyrimidine-2,4-diamine;
b) 6-[4-(4-methoxyphenyl)piperidin-1-yl]pyrimidine-2,4-diamine;
c) 2,4-dimethoxy-6-(4-phenylpiperidin-1-yl)pyrimidine;
d) 4-methyl-6-(4-phenylpiperidin-1-yl)pyrimidin-2-amine;
e) 6-[4-(4-fluorophenyl)piperidin-1-yl]pyrimidine-2,4-diamine;
f) 1-(2,6-diaminopyrimidin-4-yl)-4-[3-(trifluoromethyl)phenyl]piperidin-4-ol;
g) 6-(4-phenylpiperazin-1-yl)pyrimidine-2,4-diamine;
h) 6-[4-(4-methoxyphenyl)piperazin-1-yl]pyrimidine-2,4-diamine;
i) 6-[4-(4-fluorophenyl)piperazin-1-yl]pyrimidine-2,4-diamine;
j) 6-(3-phenylpyrrolidin-1-yl)pyrimidine-2,4-diamine;
k) 6-(4-phenylpiperazin-1-yl)pyrimidine-2,4-diamine hydrochloride;
l) 2,4-dimethoxy-6-(4-phenylpiperazin-1-yl)pyrimidine;
m) 4-methyl-6-(4-phenylpiperazin-1-yl)pyrimidin-2-amine;
n) 1-(2,6-diaminopyrimidin-4-yl)-4-phenylpiperidin-4-ol;
o) 1-(2,6-diaminopyrimidin-4-yl)-4-phenylpiperidin-4-ol hydrochloride;
p) 1-(2,6-diaminopyrimidin-4-yl)-4-(4-methoxyphenyl)piperidin-4-ol;
q) 1-(2,6-dimethoxypyrimidin-4-yl)-4-phenylpiperidin-4-ol;
r) 1-(2-amino-6-methylpyrimidin-4-yl)-4-phenylpiperidin-4-ol;
s) 1-(2-amino-6-methylpyrimidin-4-yl)-4-(4-fluorophenyl)piperidin-4-ol;
t) 1-(2,6-diaminopyrimidin-4-yl)-3-phenylpyrrolidin-3-ol;
u) 1-(2,6-diaminopyrimidin-4-yl)-3-(4-methoxyphenyl)pyrrolidin-3-ol;
v) 2,4-dimethoxy-6-(4-phenylazepin-1-yl)pyrimidine;
w) 4-methyl-6-[4-(3-chlorophenyl)azepin-1-yl)pyrimidin-2-amine;
x) 4-methyl-6-(4-phenyl-4-methoxyazepin-1-yl)pyrimidin-2-amine; and
y) 2-methyl-6-(3-phenylazetidin-1-yl)pyrimidin-4-amine;

which are shown in Table 1.

TABLE 1

| | |
|---|---|
| a) | 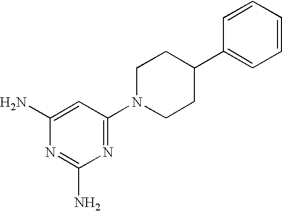 |
| b) | 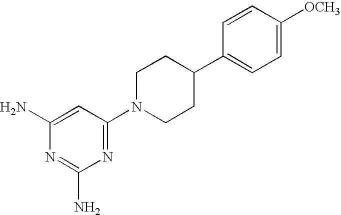 |
| c) | 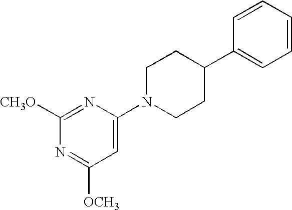 |
| d) | 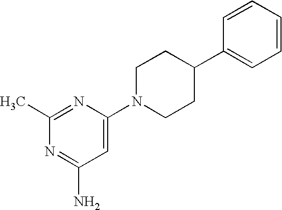 |
| e) | 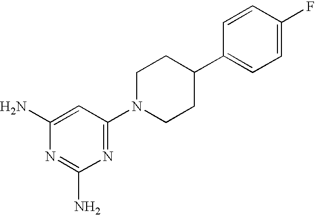 |
| f) | 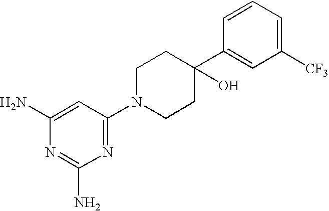 |
| g) | 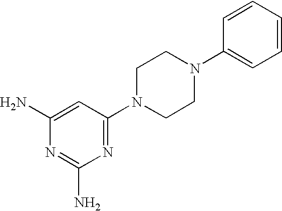 |
| h) | 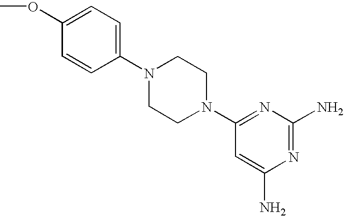 |
| i) | 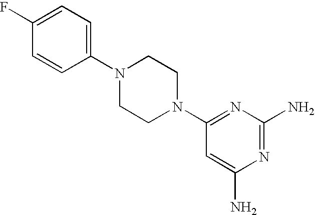 |
| j) | 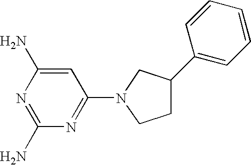 |
| k) | 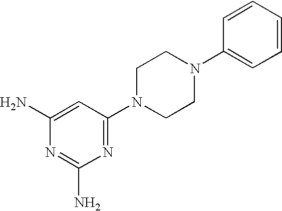 |
| l) | 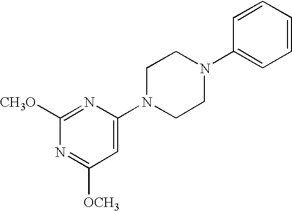 |
| m) | 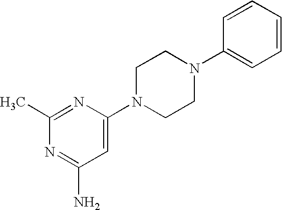 |
| n) | 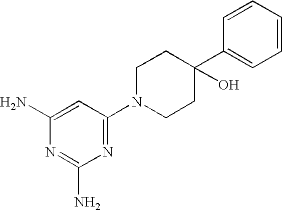 |
| o) | 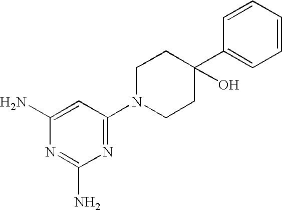 |
| p) | 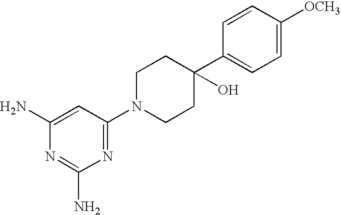 |
| q) | 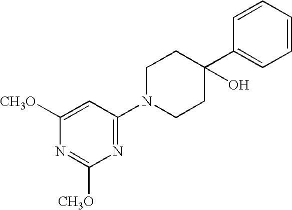 |
| r) | 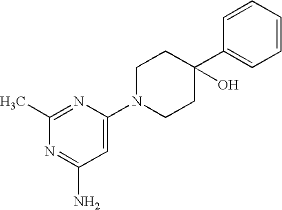 |
| s) | 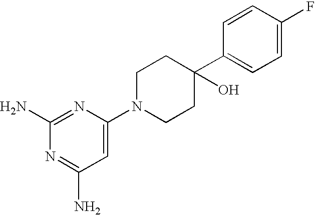 |
| t) | 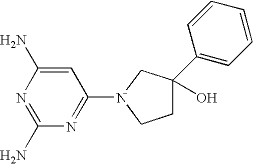 |
| u) | 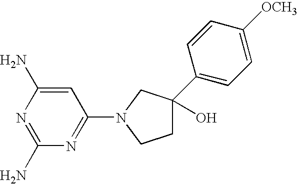 |
| v) | 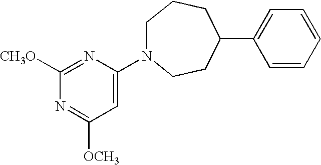 |
| w) | 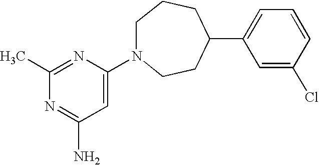 |
| x) | 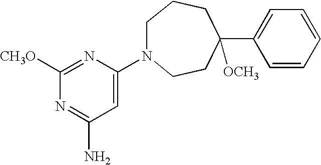 |
| y) | 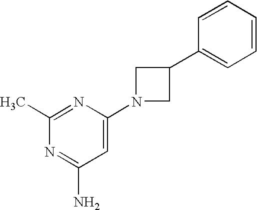 |

The following general synthetic sequence is useful in making compounds of the present invention.

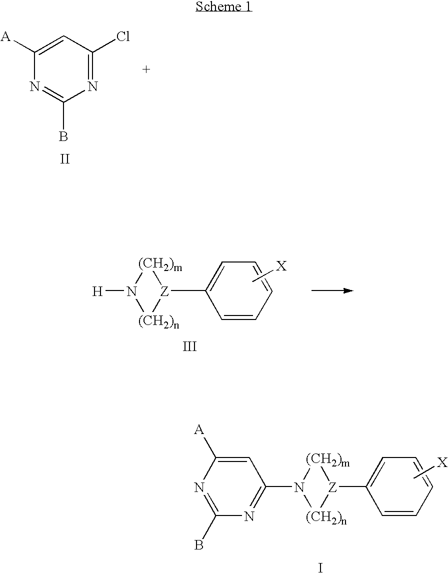

Reaction of substituted 6-chloropyrimidines II with a 4-arylcycloalkylamine of Formula III provides the compounds of the invention. The reaction is conducted in a suitable solvent such as dimethylformamide (DMF), N-methylpyrrolidone, dimethylacetamide, acetonitrile and the like, in the presence of a tertiary organic base and an alkaline earth iodide at a temperature of from 0° C. to 150° C. Non-limiting examples of alkaline earth iodides include lithium iodide, sodium iodide and potassium iodide. Non-limiting examples of tertiary organic bases include triethylamine, n-methylpiperidine, 4-dimethylaminopyridine, diazabicycloundecane and the like.

The chloropyrimidines are commercially available or can be prepared by methods described in J. Org. Chem., 1973, 38, 4386. 4-Phenyl piperidines are commercially available or can be prepared by known methods, for example those described in J. Org. Chem., 1971, 36, 522; 4-Phenylpiperazines are also commercially available or may be prepared by known methods, for example those described in Tetrahedron Lett., 1994, 34, 7331. 3-Phenylpyrrolidines are known and can be prepared, for example, by methods described in International Patents WO 01/55132 and WO 97/09328 and J. Org. Chem., 1990, 55, 270. 3-Phenylazetidines are also known and can be prepared, for example, by methods described in International Patents WO 01/07022 and WO 01/55132. 3-Phenylazepines may be prepared, for example, by methods described in U.S. Pat. No. 6,046,211.

### EXAMPLES

It is believed that one skilled in the art can, using the preceding description, practice the present invention to its fullest extent. The following detailed examples describe how to perform the various processes of the invention and are to be construed as merely illustrative and not limitations of the preceding disclosure in any way whatsoever. Those skilled in the art will promptly recognize appropriate variations from the procedures and techniques.

### Example 1

### Preparation of 2

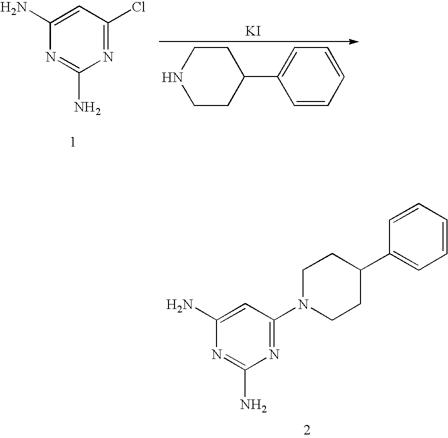

4-Chloro-2,6-diaminopyrimidine (1, 289 mg, 2 mmol), 4-phenylpiperidine (322 mg, 2.0 mmol) and potassium iodide (332 mg, 2 mmol) was heated in DMF (15 mL) at 90° C. for 16 hours. After the mixture was cooled down to room temperature (rt), water was added. The precipitate (50 mg) was collected by filtration.

Physical characteristics: MS (ES+) for m/z 270 (M+H)⁺; ¹H NMR (CDCl₃) δ 7.4-7.1, 5.20. 4.7-4.3, 2.84, 2.73, 1.9-1.6.

### Example 2

### Preparation of the Hydrochloride Salt of 2

Compound 2 (30 mg) was treated with 0.5 M HCl in MeOH (0.5 mL) at room temperature for 10 minutes. The mixture was concentrated and the residue was recrystallized from MeOH/Ether. The white precipitate (25 mg) was collected by filtration.

Physical characteristics: MS (ES+) for m/z 270 (M+H)⁺; ¹H NMR (DMSO) δ 7.5-7.2, 5.32. 3.02, 2.85, 1.9-1.5.

### Example 3

### Preparation of 4

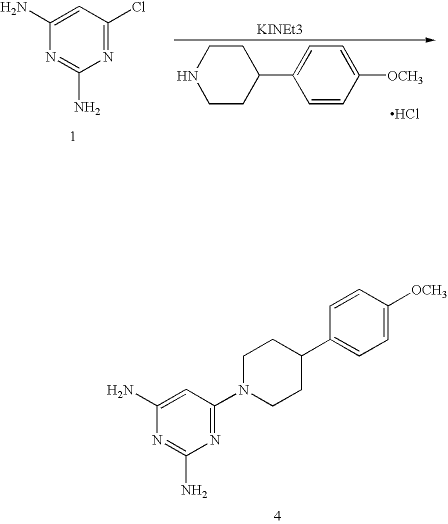

4-Chloro-2,6-diaminopyrimidine (1, 145 mg, 1 mmol), 4-(4-methoxyphenyl)piperidine hydrochloride (228 mg, 1 mmol), potassium iodide (166 mg, 1 mmol) and triethyl amine (0.3 mL, 2 mmol) were heated in DMF (10 mL) at 90° C. for 16 hours. After the mixture was cooled down to room temperature, water was added. The brown precipitate (35 mg) was collected by filtration.

Physical characteristics: MS (ES+) for m/z 300 (M+H)⁺; ¹H NMR (CDCl₃) δ 7.13, 6.86, 5.20. 4.6-4.3, 2.84, 2.69, 1.9-1.6.

### Example 4

### Preparation of 6

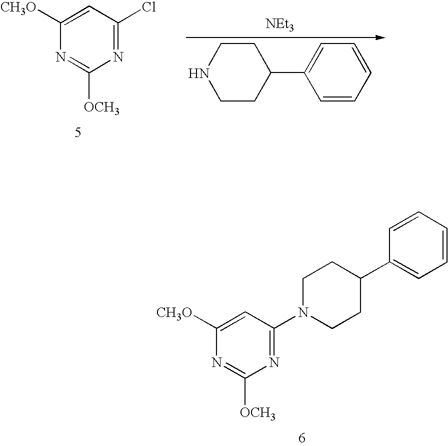

6-Chloro-2,4-dimethoxypyrimidine (5, 175 mg, 1 mmol), 4-phenylpiperidine (161 mg, 1 mmol) and triethyl amine (0.3 mL, 2 mmol) were heated in dimethylacetamide (DMA, 6 mL) at 110° C. for 1 hour. After cooling down to room temperature, the reaction mixture was poured into brine (20 mL). The mixture was then extracted with diethyl ether (20 mL). The organic layer was dried (Na₂SO₄) and concentrated. The residue was chromatographed on a silica plate by elution with 20% ethyl acetate in hexanes. The desired compound was isolated as an oil (100 mg).

Physical characteristics: MS (ES+) for m/z 300 (M+H)⁺; ¹H NMR (CDCl₃) δ 7.4-7.2, 5.55, 4.47. 3.92, 3.90, 2.93, 2.76, 2.0-1.6.

### Example 5

### Preparation of 8

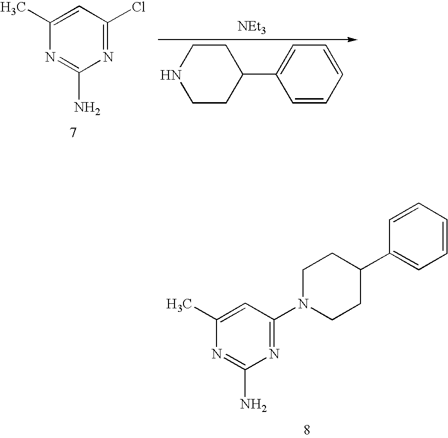

2-Amino-4-chloro-6-methylpyrimidine (7, 144 mg, 1 mmol), 4-phenylpiperidine (161 mg, 1 mmol) and triethyl amine (0.2 mL, 1.5 mmol) were heated in DMA (6 mL) at 110° C. for 1 hour. After cooling down to room temperature, the reaction mixture was poured into water (20 mL). The brown precipitate (125 mg) was collected by filtration.

Physical characteristics: MS (ES+) for m/z 269 (M+H)⁺; ¹H NMR (CDCl₃) δ 7.4-7.2, 5.90, 4.8-4.5, 2.90, 2.77, 2.23, 2.0-1.6.

### Example 6

### Preparation of 9

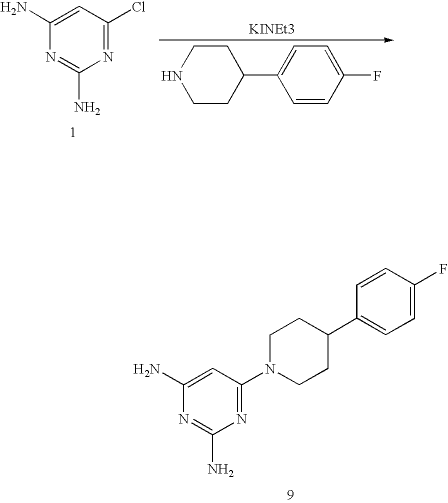

4-Chloro-2,6-diaminopyrimidine (1, 289 mg, 2 mmol), 4-(4-fluorophenyl)piperidine (358 mg, 2.0 mmol), triethyl amine (0.3 mL, 2 mmol) and potassium iodide (332 mg, 2 mmol) were heated in DMF (15 mL) at 90° C. for 16 hours. After the mixture was cooled down to room temperature, water was added. The precipitate (100 mg) was collected by filtration.

Physical characteristics: MS (ES+) for m/z 288 (M+H)⁺; ¹H NMR (CDCl₃) δ 7.2-7.0, 5.20. 4.6-4.3, 2.85, 2.69, 1.9-1.6.

### Example 7

### Preparation of 10

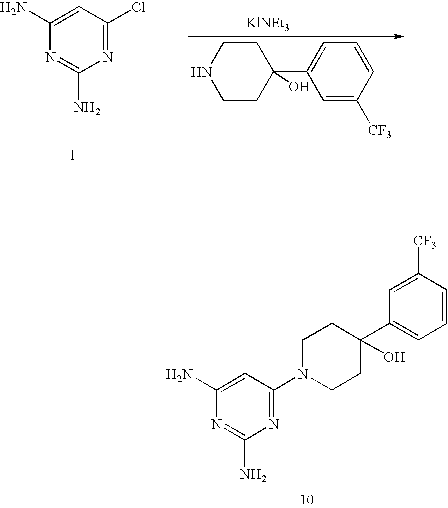

4-Chloro-2,6-diaminopyrimidine (1, 289 mg, 2 mmol), 4-(3-trifluoromethylphenyl)piperidin-4-ol (490 mg, 2.0 mmol), triethyl amine (0.3 mL, 2 mmol) and potassium iodide (332 mg, 2 mmol) were heated in DMA (dimethyl acetamide, 6 mL) at 110° C. for 4 hours. After the mixture was cooled down to room temperature, water was added. The pale yellow precipitate (100 mg) was collected by filtration.

Physical characteristics: MS (ES+) for m/z 354 (M+H)⁺; ¹H NMR (DMSO) δ 7.9-7.5, 5.66, 5.46, 5.33, 5.11, 4.07, 3.12, 1.9-1.6.

### Example 8

### Preparation of 11

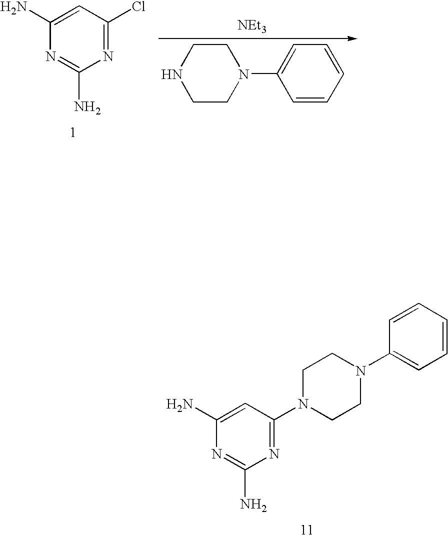

4-Chloro-2,6-diaminopyrimidine (1, 288 mg, 2 mmol), 1-phenylpiperazine (324 mg, 2 mmol) and triethyl amine (0.4 mL, 3 mmol) were heated in DMA (6 mL) at 100° C. for 2 hours. After cooling down to room temperature, the reaction mixture was poured into water (20 mL). The brown precipitate (320 mg) was collected by filtration.

Physical characteristics: MS (ES+) for m/z 271 (M+H)⁺; ¹H NMR (CDCl₃) δ 7.4-6.9, 5.19, 4.54, 4.36, 3.67, 3.23.

### Example 9

### Preparation of 12

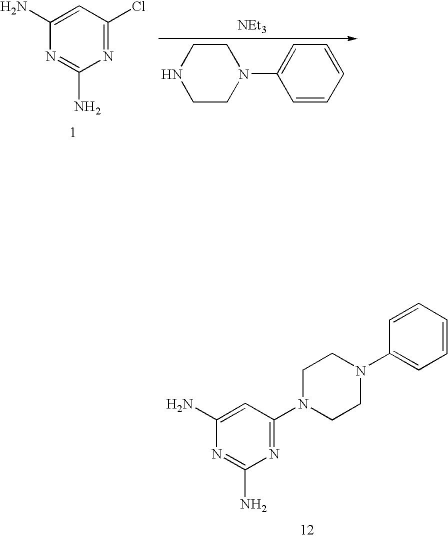

4-Chloro-2,6-diaminopyrimidine (1, 288 mg, 2 mmol), 1-phenylpiperazine (324 mg, 2 mmol) and triethyl amine (0.4 mL, 3 mmol) were heated in DMA (6 mL) at 100° C. for 2 hours. After cooling down to room temperature, the reaction mixture was poured into water (20 mL). The brown precipitate (320 mg) was collected by filtration.

Physical characteristics: MS (ES+) for m/z 271 (M+H)⁺; ¹H NMR (CDCl₃) δ 7.4-6.9, 5.19, 4.54, 4.36, 3.67, 3.23.

### Example 10

### Preparation of 13

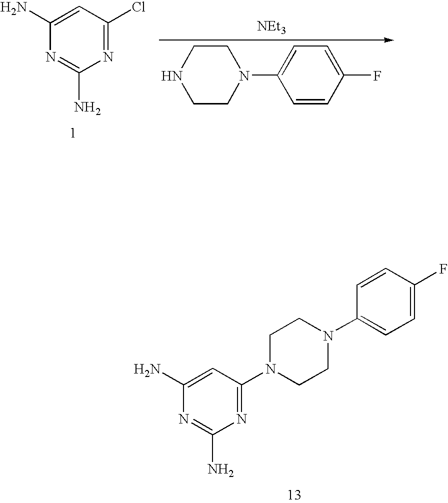

4-Chloro-2,6-diaminopyrimidine (1, 144 mg, 1 mmol), 1-(4-fluoro)phenylpiperazine (177 mg, 1 mmol) and triethyl amine (0.2 mL, 1.5 mmol) were heated in DMA (3 mL) at 100° C. for 2 hours. After cooling down to room temperature, the reaction mixture was poured into water (20 mL). The brown precipitate (150 mg) was collected by filtration.

Physical characteristics: MS (ES+) for m/z 289 (M+H)⁺; ¹H NMR (CDCl₃) δ 7.1-6.9, 5.18, 4.54, 4.37, 3.67, 3.13.

### Example 11

### Preparation of 14

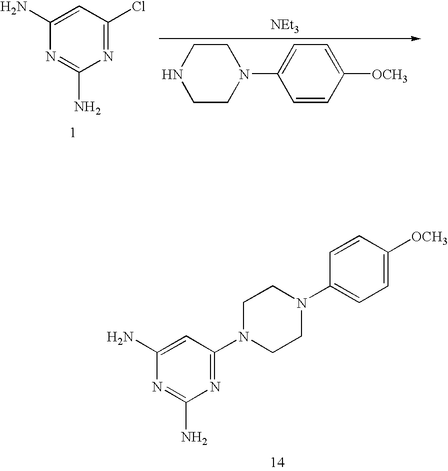

4-Chloro-2,6-diaminopyrimidine (1, 288 mg, 2 mmol), 1-(4-methoxy)phenylpiperazine (384 mg, 2 mmol) and triethyl amine (0.4 mL, 3 mmol) were heated in DMA (6 mL) at 100° C. for 2 hours. After cooling down to room temperature, the reaction mixture was poured into water (20 mL). The brown precipitate (450 mg) was collected by filtration.

Physical characteristics: MS (ES+) for m/z 301 (M+H)⁺; ¹H NMR (CD₃OD) δ 6.95, 6.86, 5.18, 3.79, 3.64, 3.10.

### Example 12

### Preparation of 15

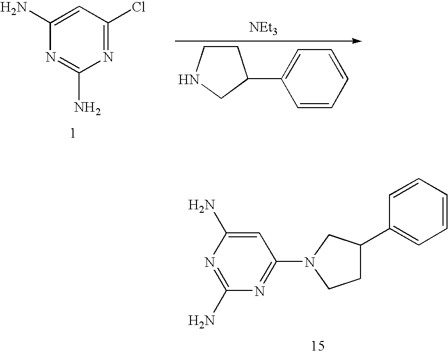

4-Chloro-2,6-diaminopyrimidine (1, 73 mg, 0.5 mmol), 3-Phenylpyrrolidine (82 mg, 0.55 mmol) and triethyl amine (0.14 mL, 1 mmol) were heated in DMA (2 mL) at 90° C. for 6 hours. After cooling down to room temperature, the reaction mixture was partitioned between methylene chloride (20 mL) and brine (20 mL). The organic layer was separated, dried (MgSO₄) and concentrated. The residue was subjected to chromatography by eleution of 5% MeOH in methylene chloride to give 15 as a white solid (20 mg).

Physical characteristics: MS (ES+) for m/z 256 (M+H)⁺; ¹H NMR (CD₃OD) δ 7.4-7.2, 5.04, 3.86, 3.64, 3.-3.4, 2.37, 2.09.

### Example 13

### Anthelmintic Activity

Compounds can be evaluated for anthelmintic activity according to the H. contortus Larval Development Assay described in Journal of Helminthology, 1984, 58, 107. In this assay, Compound 2 (6-(4-phenylpiperidin-1-yl)pyrimidine-2,4-diamine hydrochloride), at 10 μM showed inhibited motility of the larvae.

### Example 14

### Insecticidal Activity of Selected Compounds

Selected compounds were evaluated for their insecticidal activity in a binding assay as described in U.S. Pat. No. 5,859,188 (Geary, et.al., 1999). Results of the evaluations are given in Table 2 wherein % inhibition means percent displacement of a radiolabelled ligand at 25 micromolar as described.

Bead/Membrane Preparation

DAR-2 peptide (E or SRPYSFGL-NH₂) and the Drosophila type 2 allatostatin receptor (Dm4) binding studies were run utilizing a 96-well plate SPA (Scintillation Proximity Assay). A previously prepared frozen Dm4SHEP membrane preparation (with 28° C. temperature shift during growth) was used and had a protein concentration ranging from 0.547 to 1.19 mg/ml (dependent on the prep). The membranes were prepared for testing by first incubating them with WGA (wheatgerm agglutinin) SPA beads (Amersham Pharmacia Biotech RPNQ0001) in test assay buffer (20 mM Hepes, 10 mM MgCl₂, pH 7.4) for 30 minutes. Beads were initially made up at 50 mg/ml in the assay buffer and then 0.75 ml of beads were added to 375 μg of membrane and assay buffer to yield a final volume of 1.875 ml. The mixtures were incubated at room temperature for 30 minutes with occasional shaking (inverting several times every 5 minutes) followed by centrifugation at 1200 RPM for 10 minutes. The supernatant was removed and the beads resuspended in a total volume of 50 ml by addition of assay buffer to give a final concentration of 0.15 μg membrane per 20 μl.

Binding Assay

96-well plates used in the SPA were Wallac 1450-401. All treatments were run in duplicate. Unknowns were evaluated as follows. To each well a total volume of 100 μl was added. The contents, in the order added, were 50 μl (or 60 μl for total binding replicates) assay buffer (20 mM Hepes, 10 mM MgCl₂, pH 7.4), 10 μl of test compound, 20 μl of the previously described [¹²⁵I]SRPYSFGL-NH₂ and 20 μl of the previously described membrane preparations (0.15 μg). DMSO was used to solubilize unknowns and had a final concentration in the well of 1%. Concentration of iodinated ligand used was 0.08 nM (the determined K_{d} concentration, see below). All unknowns were tested at an initial concentration of 10 μM for their ability to block [¹²⁵I]SRPYSFGL-NH₂ binding to the receptor, If required, these were subsequently titrated at 10, 1, 0.1, 0.01, 0.001, 0.0001, 0.00001 and 0.000001 μM in order to calculate IC₅₀ values. Total binding (TB) replicates and non-specific binding (NSB) replicates were run as standards in each test. TB wells contained no blocker (buffer only) and NSB wells contained a final concentration of 10 μM SRPYSFGL-NH₂. Specific binding was typically 93% or better. NSB was subtracted from all binding values prior to further analysis. In all testing a standard IC₅₀ curve for SRPYSFGL-NH₂ (at the concentrations listed above for unknowns) was run and analyzed for comparative purposes. IC₅₀ values for SRPYSFGL-NH₂ were typically 0.01 nM. Additional studies were run previous to testing unknowns and included saturation binding (SB) experiments. SB studies included 8 iodinated ligand concentrations (0.004 to 0.4 nM) with determined K_{d} and Bₘₐₓ values of 0.081 nM and 10.02 pM/mg bound, respectively. Following replicate preparation, test plates were placed on a platform shaker (20 rpm) for 5 minutes and then counted on a Wallac 1450 Microbeta counter continuously over a 20 hour period. Data from 12 hours post membrane addition was used for analysis. Prism 2.0 and 3.0 (Graphpad Inc.) were used for all data analysis.

Selected compounds were evaluated for their insecticidal activity in the binding assay as described above. Results of the evaluations are given in Table 2.

TABLE 2

| | IC50 |
|---|---|
| Compound | (μM) |
| 2 | 0.79 |
| 3 | 0.2-0.4 |
| 4 | 1.6 |
| 6 | 222 |
| 8 | 2.64 |

Having described the invention in detail and by reference to the embodiments thereof, it will be apparent that modifications and variations are possible without departing from the scope of the appended claims.

1. A compound of Formula I: 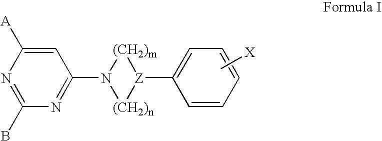
wherein A and B are independently selected from the group comprising H, C₁₋₆ alkyl, NRR₁, and C₁₋₆ alkoxy;
X is one to three substituents selected from the group comprising halogen, C₁₋₆ alkyl, —OH, C₁₋₆ alkoxy, C₁₋₆ thioalkoxy, —NRR₁, —CO₂R, —CONRR₁, —CN, —NO₂ and —SO₂NRR₁;
R and R₁ are independently H, C₁₋₆ alkyl or, taken together with the N to which they are attached form a 5-7 membered ring optionally containing an additional heteroatom of NR, O or S;
m is 1 or 2;
n is 1, 2 or 3;
when m and n are both 2, Z is N, CH or C—O—R;
when m and n are not both 2, Z is CH or C—O—R;
or pharmaceutically acceptable salts thereof.

2. A compound according to claim 1 selected from the group:
6-(4-phenylpiperidin-1-yl)pyrimidine-2,4-diamine;
6-[4-(4-methoxyphenyl)piperidin-1-yl]pyrimidine-2,4-diamine;
2,4-dimethoxy-6-(4-phenylpiperidin-1-yl)pyrimidine;
4-methyl-6-(4-phenylpiperidin-1-yl)pyrimidin-2-amine;
6-[4-(4-fluorophenyl)piperidin-1-yl]pyrimidine-2,4-diamine;
1-(2,6-diaminopyrimidin-4-yl)-4-[3-(trifluoromethyl)phenyl]piperidin-4-ol;
6-(4-phenylpiperazin-1-yl)pyrimidine-2,4-diamine;
6-[4-(4-methoxyphenyl)piperazin-1-yl]pyrimidine-2,4-diamine;
6-[4-(4-fluorophenyl)piperazin-1-yl]pyrimidine-2,4-diamine;
6-(3-phenylpyrrolidin-1-yl]pyrimidine-2,4-diamine;
6-(4-phenylpiperazin-1-yl)pyrimidine-2,4-diamine hydrochloride;
2,4-dimethoxy-6-(4-phenylpiperazin-1-yl)pyrimidine;
4-methyl-6-(4-phenylpiperazin-1-yl)pyrimidin-2-amine;
1-(2,6-diaminopyrimidin-4-yl)-4-phenylpiperidin-4-ol;
1-(2,6-diaminopyrimidin-4-yl)-4-phenylpiperidin-4-ol hydrochloride;
1-(2,6-diaminopyrimidin-4-yl)-4-(4-methoxyphenyl)piperidin-4-ol;
1-(2,6-dimethoxypyrimidin-4-yl)-4-phenylpiperidin-4-ol;
1-(2-amino-6-methylpyrimidin-4-yl)-4-phenylpiperidin-4-ol;
1-(2-amino-6-methylpyrimidin-4-yl)-4-(4-fluorophenyl)piperidin-4-ol;
1-(2,6-diaminopyrimidin-4-yl)-3-phenylpyrrolidin-3-ol;
1-(2,6-diaminopyrimidin-4-yl)-3-(4-methoxyphenyl)pyrrolidin-3-ol;
2,4-dimethoxy-6-(4-phenylazepin-1-yl)pyrimidine;
4-methyl-6-[4-(3-chlorophenyl)azepin-1-yl)pyrimidin-2-amine;
4-methyl-6-(4-phenyl-4-methoxyazepin-1-yl)pyrimidin-2-amine;
2-methyl-6-(3-phenylazetidin-1-yl)pyrimidin-4-amine; and
pharmaceutically acceptable salts thereof.

3. A method for preventing or treating parasitic diseases in a mammal comprising treating said mammal with an effective amount of one to three compounds selected from the compounds of claim 1.

4. The method according to claim 3 wherein said mammal is selected from the group of humans, cattle, sheep, deer, horses, dogs, cats, goats, swine, and birds.

5. A method for preventing or treating parasitic diseases in a mammal comprising treating said mammal with an effective amount of one to three compounds selected from the compounds of claim 2.

6. The method according to claim 5 wherein said mammal is selected from the group of humans, cattle, sheep, deer, horses, dogs, cats, goats, swine, and birds.

7. A method for producing a medicament useful for preventing or treating parasitic diseases in a mammal comprising the use of one to three compounds selected from the compounds of claim 1.

8. A method for producing a medicament useful for preventing or treating parasitic diseases in a mammal comprising the use of one to three compounds selected from the compounds of claim 2.

9. A composition comprising one to three compounds of claim 1.

10. A composition comprising one to three compounds of claim 2.

11. A method for the prevention or treatment of parasites in a plant or agricultural crop which comprises treating said plant or crop, or the soil in which they grow, with an effective amount of one to three compounds of claim 1.

12. A method for the prevention or treatment of parasites in a plant or agricultural crop which comprises treating said plant or crop, or the soil in which they grow, with an effective amount of one to three compounds of claim 2.
